(19) United States
(12) Patent Application Publication
(10) Pub. No.: US 2005/0004016 A1
(43) Pub. Date: Jan. 6, 2005

(54) Method of obtaining peptides with tissue-specific activity and pharmaceutical compositions on their basis

(76) Inventor: Vladimir Khatskelevich Khavinson, St. Petersburg (RU)

Correspondence Address: LEYDIG VOIT & MAYER, LTD, 700 THIRTEENTH ST. NW, SUITE 300, WASHINGTON, DC, 20005-3960, US

(21) Appl. No.: 10/312,291

(22) PCT Filed: Jun. 26, 2001
(86) PCT No.: PCT/RU01/00256

(30) Foreign Application Priority Data
Jun. 29, 2000 (RU) 2000116791

(51) Int. Cl. A61K 38/17; G01N 33/53; G01N 33/567

(52) U.S. Cl. 514/12; 435/7.2

(57) ABSTRACT

The invention refers to the field of chemistry and concerns the method of obtaining peptides with tissue-specific activity by targeted chemical synthesis. This invention can be employed in medicine to obtain peptide-based pharmaceuticals normalising the functions of various organs and functions. The method of obtaining peptides proposed in this patent claim embraces quantitative amino acid analysis of acetic extracts from tissues, selection on its basis of two amino acids (Glu and Asp) prevailing in the studied tissue, synthesis of the central link from these amino acids and attachment to its N- and C-ends of the amino acids prevailing among the remaining amino acids in the studied tissue. The peptides obtained by the claimed method possess a tissue-specific activity. There is proposed a pharmaceutical composition possessing a tissue-specific activity and containing as its active base one of the peptides obtained by the claimed method or its salts and a pharmaceutically admissible carrier.

### FIELD OF THE INVENTION

The invention refers to the field of chemistry and concerns the method of obtaining peptides with tissue-specific activity by targeted chemical synthesis. The invention can be employed in medicine to obtain pharmaceuticals based on these peptides normalising the functions of various organs and tissues.

### BACKGROUND ON THE INVENTION

There are known the methods of obtaining complex peptide substances with tissue-specific activity: Thymalin (1), Epithalamin (2), Prostatilen (3), Cortexin (4) and Retinalamin (5). These substances are complexes of low-molecular polypeptides obtained from animal organs and tissues by extraction in 3% acetic add with chlorous zinc and further treatment of the supraprecipitation fluid with acetone (6). The given method of obtaining peptide substances is marked by considerable variability of physical and chemical properties of the exacted peptides and presence of ballast components in them. Limited reserves of required organic raw material and high labour and energy consumption by production are the drawbacks of the said method of obtaining complex peptide substances, which impede their industrial production.

There are known the methods of obtaining peptides by classical peptide synthesis in a solution (7) on the basis of active fractions isolated from Thymalin—Glu-Trp (Thymogen) (8), Thymopoietin II—Arg-Lys-Asp-Val-Tyr (Thymopentin) (9), Splenin—Arg-Lys-Glu-Val-Tyr (Splenopentin) (10), Immunoglobulin G—Thr-Lys-Pro-Arg (Taphcin) (11) and other. However, the substances obtained by the given method primarily possess a singly directed spectrum of biological activity (immunoregulatory), instability of the substances in a solution and high doses of application.

There are known methods of obtaining peptides of modified structure by classical peptide synthesis in a solution (7): Arg-α-Asp-Lys-Val-Tyr-Arg (Immunophan) (12) structurally distinguished from Thymopentin by the presence of amino acid substitutes with the chain elongated by end Arginine; γ-Glu-Trp (Bestim) (13) structurally distinguished from Thymogen by γ-bond. These substances are also characterised by their narrowly directed immunobiological activity and complicated procedure of chemical synthesis.

There is also known a synthetic polymer (Cop 1) inhibiting cellular immune response, which is obtained by chemical synthesis and contains amino acids L-Ala, L-Glu, L-Lys, L-Tyr in the following molar correlation: 6.0:1.9:4.7:1.0 (14). This method of obtaining active substances is not widespread and concerns only the design of polymer Cop 1.

### DISCLOSURE OF THE INVENTION

This invention operates the following specific terminology accepted in this field.

The notion “small regulatory peptides” under this patent claim implies the presence of endogenous small peptides formed in the process of proteolysis and their synthetic analogues of the known amino acid sequence (up to 10 amino acids), which reveal physiologically active properties (7, 15).

The notion “tissue-specific activity” of peptides under this patent claim implies the impact of peptides on the very tissues, whose amino acid composition serves the basis for their acquisition (6, 16, 17).

The notion “pharmaceutical composition” under this patent claim implies an active peptide obtained by the claimed method or its salts of the amino group, of carboxyl groups, salts of organic and inorganic cations and a pharmacologically admissible carrier.

The notion “effective quantity” under this patent claim implies the employment of such an amount of the active base, which, in compliance with the quantitative indices of its activity and toxicity, as well as with respect to the knowledge available, shall be effective in a given drug form.

The purpose of this patent claim consists in creating a method of obtaining peptides with tissue-specific activity by targeted chemical synthesis, which enables its industrial employment at minimal costs, as well as in creating pharmaceutical compositions exerting a tissue-specific effect and based on the peptides obtained by the claimed method.

The claimed method embraces quantitative amino acid analysis of acetic extracts from animal tissues, selection on its basis of two amino acids (Glu and Asp) prevailing in the studied tissue, synthesis of the central link from these amino acids and attachment to its N- and C-ends of the amino acids prevailing among the remaining amino acids in the studied tissue.

Peptides obtained by the claimed method possess a tissue-specific activity, i.e. they exert an activity upon the very tissues whose amino acid composition serves the basis for their acquisition.

This patent claim describes a pharmaceutical composition of tissue-specific activity containing as its active base an effective quantity of one of the peptides obtained by the claimed method or its salts of the amino acid group, of carboxyl groups, salts of organic and inorganic cations and a pharmaceutically admissible carrier, for example, isotonic sodium chloride solution.

To obtain pharmaceutical compositions meeting the invention, the proposed peptides or their pharmaceutically admissible carriers in the form of salts are blended as active bases with a pharmaceutically admissible carrier according to the methods of compounding accepted in pharmaceutics. The carrier may have various forms depending on the drug form preferable for administration to a body.

### INDUSTRIAL APPLICATION

The proposed invention is illustrated by the examples of:
amino acid analysis of acetic extracts from the tissues of the epiphysis, cerebral cortex, brain and liver (Example 1);
synthesis of Ala-Glu-Asp-Gly tetrapeptide (Example 2);
effect of Ala-Glu-Asp-Gly tetrapeptide on the growth of brain subcortical structure explants (Example 3) clearly demonstrating the revealed tissue-specific properties;
synthesis of Ala-Glu-Asp-Pro tetrapeptide (Example 4);
effect of Ala-Glu-Asp-Pro tetrapeptide on the growth of cerebral cortex explants (Example 5) clearly demonstrating the revealed tissue-specific properties;
synthesis of Lys-Glu-Asp-Ala tetrapeptide (Example 6);
effect of Lys-Glu-Asp-Ala tetrapeptide on the protein synthesis intensity in a monolayer hepatocyte culture of rats of different age (Example 7) clearly demonstrating the revealed tissue-specific properties;
effect of Lys-Glu-Asp-Ala tetrapeptide on the growth of liver explants (Example 8) clearly demonstrating the revealed tissue-specific properties.

### EXAMPLE 1

### Amino Acid Analysis of Acetic Extracts from the Tissues of Epiphysis, Cerebral Cortex and Liver

TABLE 1
Amino acid analysis of acetic extracts from the tissues
of epiphysis, cerebral cortex and liver

| | Relative content | | |
|---|---|---|---|
| Amino acids | Epiphysis | Cerebral cortex | Liver |
| Alanine | 1.42** | 1.90** | 2.31** |
| Arginine | 0.84 | 1.15 | 0.75 |
| Asparagine | — | — | — |
| Aspartic acid | 1.66* | 2.70* | 2.45* |
| Valine | 1.29 | 1.01 | 1.28 |
| Histidine | 0.61 | 0.62 | 0.81 |
| Glycine | 1.39*** | 1.38 | 1.65 |
| Glutamine | — | — | — |
| Glutamic acid | 2.96* | 4.37* | 2.73* |
| Isoleucin | 0.36 | 0.60 | 0.32 |
| Leucin | 1.06 | 1.00 | 1.25 |
| Lysine | 1.29 | 1.77 | 2.19** |
| Methionine | 0.22 | 0.37 | 0.34 |
| Proline | 0.95 | 2.07*** | 1.04 |
| Serine | 1.05 | 1.33 | 1.20 |
| Threonine | 0.68 | 0.82 | 0.78 |
| Tryptophan | — | — | — |
| Tyrosine | 0.36 | 0.60 | 0.33 |
| Phenyl alanine | 0.46 | 0.54 | 0.61 |
| Cyctein | — | — | — |

*amino acids prevailing in the studied tissue, of which the peptide central link is synthesised;
**amino acids joined to the central link at the N-end;
***amino acids joined to the central link at the C-end.

Amino acid analysis of acetic extracts from animal tissues enabled to reveal four prevailing amino acids for each tissue. The choice of this very number of amino acids was determined by the fact that lower number of amino acids (e.g., two or three) would inevitably result in an amino acid set with low specificity to the given tissue. Thus, the four chosen amino acids appeared sufficient to assess the minimal variability of the composition.

When modelling the proposed tissue-specific tetrapeptides we also took into account the location of hydrophobic and hydrophilic amino acid residues in a number of known tissue oligopeptides (18) corresponding to the general formula H-X-Glu-Asp-Y-OH. In this case, the partial negative charge attains its maximal possible concentration in the molecule centre. If X and Y are aliphatic hydrophobic amino acids (Ala, Gly, Pro) this site will also be the most hydrophilic. This conforms to the data evidencing that intracellular proteinases are marked by a certain preference of the bonds formed by hydrophobic amino acids (19). Another extreme case is provided for X-Lys variant. Here, the partially positive charge reaches its maximal possible concentration at the N-end of the molecule (pointing at increased hydrophilic nature as well). At the same time, this secures the potentiality of forming intramolecular quasi-cyclic structures due to electrostatic interaction of ionised γ- and β-COO(−) groups of Glu or Asp, respectively, with ε- or α-NH₃(+)- group of Lys. Presence of Pro at the C-end considerably raises the hydrophobic property of this site in comparison with Gly or Ala variants.

Consequently, selected were the following sequences of amino acids, which made a part of the tetrapeptides designed on the basis of amino aced analysis of acetic extracts from the tissues of the epiphysis, cerebral cortex and liver:
H-Ala-Glu-Asp-Gly-OH;
H-Ala-Glu-Asp-Pro-OH;
H-Lys-Glu-Asp-Ala-OH.

### EXAMPLE 2

### Synthesis of Ala-Glu-Asp-Gly Tetrapeptide

1. Product name: alanyl-glutamyl-aspartyl-glycine. 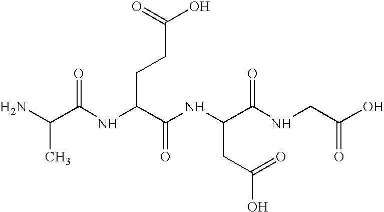
2. Structural formula: H-Ala-Glu-Asp-Gly-OH.
3. Molecular formula without ion pair: C₁₄H₂₂N₄O₉.
4. Molecular weight without ion pair: 390.35.
5. Ion pair: acetate.
6. Appearance: white amorphous powder without smell

7. Method of synthesis: the peptide is obtained by a classical method of synthesis in a solution by scheme A:

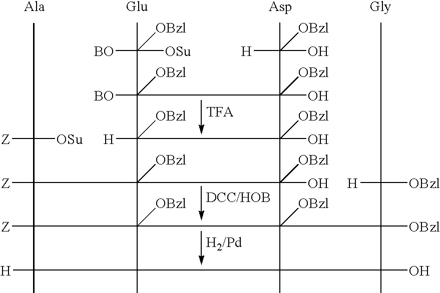

| | |
|---|---|
| Z | -benzyloxycarbonyl group; |
| BOc | -tert.butyloxycarbonyl group; |
| OSu | -N-oxysuccinimide ester; |
| OBzl | -benzyl ester; |
| DCC | -N,N′-dicyclohexylcarbodiimide; |
| HOBT | -N-oxybenzotriazol. |

N,N′-dimethylformamide was used as a solvent. When adding aspartic acid, the defence of α-COOH group was applied by salification with triethylamine. BOC-protecting group was removed with trifluoracetic acid (TFA) solution and Z-protecting group—with catalytic hydrogenation. The product was extracted and purified by the method of preparative high-performance liquid chromatography (HPLC) on a reversed phase column.

Properties of the finished product:

| | | | | |
|---|---|---|---|---|
| amino acid analysis | Glu | Asp | Ala | Gly |

| | | | |
|---|---|---|---|
| 1.02 | 1.00 | 1.01 | 1.00 |

peptide content 98.45% (by HPLC, 220 nm);
thin layer chromatography (TLC)—individual, R_{f}=0.73 (acetonitrile acetic acid-water 5:1:3);
moisture content: 5%;
pH of 0.001%-solution: 4.37;
specific rotary power [α]_{D}²² : −32° (c=1, H₂O)
“Polamat A”, Carl Zeiβ Jena.
   Example of Synthesis:
   1. BOC-Glu (OBzl)-Asp(OBzl)-OH (I), N-tert-butyloxycarbonyl-(γ-benzyl)glutamyl-(β-benzyl)aspartate.

4.34 g (0.0100 mole) of N-oxysuccinimide ester of N-tert.butyloxycarbonyl-(γ-benzyl)glutamic acid (BOC-Glu (OBzl)-OSu) is dissolved in 20 ml of dimethylformamide; and added 1.72 ml (0.0125 mole) of triethylamine and 2.80 g (0.0125 mole) of β-benzyl aspartate. The mixture is stirred for 24 hours at room temperature. Afterwards the product is precipitated with 0.5N sulphuric acid solution (150 ml), extracted by ethyl acetate (3×30 ml), washed in 0.5N sulphuric acid solution (2×20 ml), water, 5% sodium bicarbonate solution (1×20 ml), water, 0.5N sulphuric acid solution (2×20 ml), water. The product is dried over anhydrous sodium sulphate. Ethyl acetate is filtered out and removed in vacuo at 40° C. The residue is dried in vacuo over P₂O₅. 5.68 g (≈100%) of oil is obtained. R_{f}=0.42 (benzene-acetone 2:1; Sorbfil plates, Silicagel 8-12 μm, development by UV and chlorine/benzidine).

2. TFA.H-Glu(OBzl)-Asp(OBzl)-OH (II), trifluoracetate of (γ-benzyl)-glutamyl-(β-benzyl)aspartate.

5.68 g (≈0.01 mole) of N-tert.butyloxycarbonyl-(γ-benzyl)-glutamyl-(β-benzyl) aspartate (I) is dissolved in 20 ml of dichlormethan-trifluoracetic acid mixture (3:1). Two hours later the solvent is removed in vacuo at 40° C. The removal is repeated with another portion of dichlormethan (2×10 ml). The residue is dried in vacuo over NaOH. 5.80 g (≈100%) of oil is obtained. R_{f}=0.63 (n-butanol-pyridine-acetic acid-water, 15:10:3:12).

3. Z-Ala-Glu-(OBzl)-Asp(OBzl)-OH (III), N-carbobenzoxyalanyl-(γ-benzyl)-glutamyl-(β-benzyl)aspartate.

5.65 g (0.01 mole) of trifluoracetate of (γ-benzyl)-glutamyl-(β-benzyl)aspartate (II) is dissolved in 10 ml of dimethylformamide and added 2.80 ml (0.02 mole) of triethylamine and 4.14 g (0.013 mole) N-oxysuccinimide ester of N-carbobenzoxyalanine. The mixture is stirred for 24 hours at room temperature. The product is precipitated with 0.5N sulphuric acid solution (150 ml), extracted by ethyl acetate (3×30 ml), washed in 0.5N sulphuric acid solution (2×20 ml), water, 5% sodium bicarbonate solution (1×20 ml), water, 0.5N sulphuric acid solution (2×20 ml), water. The product is dried over anhydrous sodium sulphate. Ethyl acetate is filtered out and removed in vacuo at 40° C. The residue is crystallised in the ethyl acetate/hexane system. The product is filtered and dried in vacuo over P₂O₅. The yield is 4.10 g (66%). The temperature of melting (Tₘₗ) equals 154° C. R_{f}=0.48 (benzene-acetone, 1:1), R_{f}=0.72 (N-butanol-pyridine-acetic acid-water, 15:10:3:12).

4. Z-Ala-Glu-(OBzl)-Asp(OBzl)Gly-OBzl (III), N-carbobenzoxyalanyl-(γ-benzyl)glutamyl-(β-benzyl)aspartylglycine benzyl ester.

1.01 g (3 mmole) of glycine benzyl ether tosylate (TosOH.H-Gly-OBzl) is suspended in 15 ml of tetrahydrofuran and added 0.4 ml (3 mmole) of triethylamine while stirring. In 5 minutes 1.28 g (2 mmole) of N-carbobenzoxyalanine-(γ-benzyl)glutamyl-(β-benzyl) aspartate (III) and 0.27 g (2 mmole) of N-oxybenzotriazo, are added and the mixture is cooled down to 0° C. 0.42 g (2 mmole) of N,N′-dicyclohexylcarbodiimide solution cooled down to 0° C. is added in 5 ml of tetrahydrofuran. The mixture is stirred at this temperature for 2 hours and left to blend for a night at room temperature. Precipitate of dicyclohexylurea is filtered out, the solvent is removed in vacuo and the residue is dissolved in 30 ml of ethyl acetate. The product is washed in 1N hydrochloric acid solution, water, 5% sodium bicarbonate solution, water, 1N hydrochloric acid solution, water and dried over anhydrous sodium sulphate. The solvent is removed in vacuo and the product is crystallised in the ethyl acetate/hexane system. The yield is 1.30 g (82%). Tₘₗ=146-148° C. R_{f}=0.75 (benzene-acetone, 2:1).

5. H-Ala-Glu-Asp-Gly-OH (IV), alanyl-glutamyl-aspartyl-glycine.

1.25 g of benzyl ester of N-carbobenzoxyalanyl-(γ-benzyl)glutamyl-(β-benzyl)aspartylglycine (III) is hydrogenated in the methanol/water/acetic acid system (3:1:1) over Pd/C catalyst. Completeness of the deblocking reaction is monitored by TLC in the benzene/acetone (2:1) and acetonitrile/acetic acid/water (5:1:3) systems. When the reaction is over the catalyst is filtered out, the filtrate is removed in vacuo and the residue is crystallised in the water/methanol system. The product is dried in vacuo over KOH. The yield is 520 mg (95%). R_{f}=0.73 (acetonitrile-acetic acid-water, 5:1:3).

For purification, 390 mg of the preparation is dissolved in 4 ml of 0.01% trifluoracetic acid and subjected to HPLC on a reversed phase column measuring 50×250 mm (Diasorb-130-C16T, 7μ). The employed chromatograph is Beckman System Gold, 126 Solvent Module, 168 Diode Array Detector Module. Conditions of chromatography A: 0.1% of TFA; B: 50% of MeCN/0.1% of TFA, grad. B 0→5% in 80 min. Sample volume is 5 ml, detection is conducted by 215 nm, scanning—by 190-600 nm, flow rate equals 10 ml/rin. The fraction is selected within 54.0-66.0 min. The solvent is removed in vacuo at a temperature not exceeding 40° C. The removal is multiply repeated (5 times) with 10 ml of 10% acetic acid solution. The residue is finally dissolved in 20 ml of deionised water and lyophilised. 290 mg of purified preparation in the form of amorphous odourless white powder is obtained.

The obtained peptide in the form of acetate is converted into a free form by treating it with IRA anionite or its analogue in (OH⁻)-form. Afterwards salts of the amino acid group are obtained by adding an equivalent of the respective acid hydrochloric or oxalic). The obtained aqueous solution is lyophilised and analysed as a finished product.

In order to obtain corresponding salts of carboxyl groups, the free tetrapeptide is added a calculated quantity of the aqueous solution of a corresponding metal hydroxide (NaOH, KOH, Zn(OH)₂, LiOH, Ca(OH), Mg(OH)₂, NH₄OH). To obtain triethylammonium salt, the processing is carried out similarly, triethylamine being used as the base.

6. Analysis of the Finished Product.

Content of the active base (peptide) is defined by HPLC on Supelco LC-18-DB column, 4.6×250 mm, grad. LC-18-DB. A: 0.1% of TFA; B: 50% of MeCN/0.1% of TFA; grad. B 0→20% in 20 min. The flow rate equals 1 ml/min. Detection by 220 nm, scanning—by 190600 nm, the sample volume is 20 μl. Peptide content—98.5%.

The amino acid content is defined on an analyser after 24-hour hydrolysis in 6N HCl at 125° C.

| Glu | Asp | Ala | Gly |
|---|---|---|---|
| 1.02 | 1.00 | 1.01 | 1.00 |

TLC: individual, R_{f}=0.73 (acetonitrile-acetic acid-water, 5:1:3). Sorbfil plates, 8-12 μm Silicagel, developing in chlorine/benzidine.
Moisture content: 5% (gravimetrically, according to the mass loss by drying, −20 mg at 100° C.
pH of 0.001% solution: 4.37 (potentiometrically).
Specific rotary power: [α]_{D}²²: −32° (c=1, H₂O)
“Polamat A”, Carl Zeiβ Jena.

The pharmaceutical peptide substance in injection form containing the tetrapeptide as its active base is obtained the following way: the tetrapeptide obtained by the above-described method is dissolved in 0.9% isotonic sodium chloride solution. One vial contains 1 ml of the tetrapeptide solution in the concentration of 10 μg/ml.

### EXAMPLE 3

### Effect of Ala-Glu-Asp-Gly Tetrapeptide on the Growth of Brain Subcortical Structure Explants

Experiments were conducted on 69 fragments of brain subcortical structures of 10-11-days' old chicken embryos. The nutrient medium for cultivation consisted of 35% Eagle's solution, 25% calf foetal serum, 35% Hanks' solution, 5% chicken embryonic extract. The medium was also added glucose (0.6%), insulin (0.5 unit/ml), penicillin (100 unit/ml), glutamine (2 mM). Fragments of the brain subcortical structures were placed in this medium and cultivated in Petri's dishes in a thermostat at 36.7° C. for 48 hours. The experimental medium was added Ala-Glu-Asp-Gly tetrapeptide and Epithalamin in the concentrations of 2, 10, 20, 50, 100, 200, 400 ng/ml. Square index (SI) considered the criterion of biological activity. It was counted as the ratio between the total explant square including the growth zone and the initial square of the subcortical structure fragment Significance of differences between the average values of SI was assessed by Student's t-criterion. ST values were expressed percent, the control SI value being taken for 100%.

The growth zone of the control explants of the brain, subcortical structures included short neurites, migrating glia and fibroblast-resembling cells.

Immediate influence of the substances upon the fragments of the brain subcortical structures was investigated in the following experimental series.

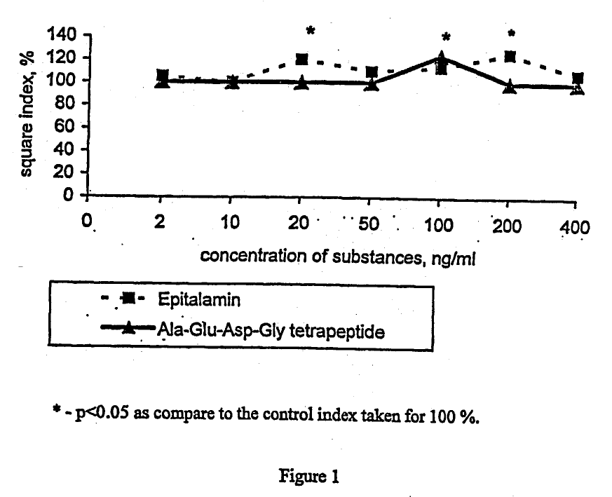

The nutrient medium of the explants of chicken embryonic subcortical structures was added Epithalamin in various concentrations. On the third day of cultivation, in the concentrations of 20 and 200 ng/ml a significant rise in the explant SI was noted in comparison with the control SI values (by 20% and 26% respectively). FIG. 1 demonstrates the effect of Ala-Glu-Asp-Gly tetrapeptide on the growth of the brain subcortical structure explants. No significant values of the subcortical structure SI were registered in other Epithalamin concentrations. Pronounced stimulation of the development of the brain subcortical structure explants was revealed by applying Ala-Glu-Asp-Gly tetrapeptide in the concentration of 100 ng/ml when SI of the experimental explants was higher by 24% than that of the control fragments.

Investigation of the subcortical structure explants by longer periods of cultivation—up 5 to 7 days—demonstrated analogous neurite stimulating effects in the same concentrations. In certain cases, a statistically insignificant decrease in explant SI was noted, probably, due to the reckon of nerve fibres by longer cultivation periods.

### EXAMPLE 4

### Synthesis of Ala-Glu-Asp-Pro Tetrapeptide

1. Product name: L-alanyl-L-glutamyl-L-aspartyl-L-proline.
2. Structural formula: H-Ala-Glu-Asp-Pro-OH. 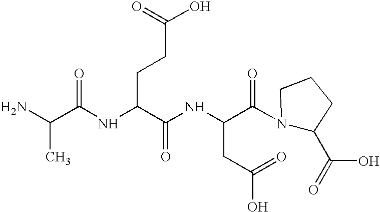
3. Gross formula without ion pair: C₁₇H₂₆N₄O₉.
4. Molecular weight without ion pair: 430.41.
5. Ion pair: acetate.
6. Appearance: white amorphous odourless powder.

7. Method of synthesis: the peptide is obtained by a classical method of synthesis in a solution by Scheme B.

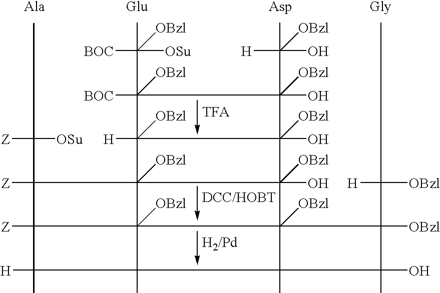

| | |
|---|---|
| Z | -benzyloxycarbonyl group; |
| BOC | -tert.butyloxycarbonyl group; |
| OSu | -N-oxysuccinimide ester, |
| OBzl | -benzyl ester; |
| DCC | -N,N′-dicyclohexylcarbodiimide; |
| HOBT | -N-oxybenzotriazol. |

N,N′-dimethylformamide was used as a solvent. When adding aspartic acid, the defence of α-COOH group was applied by salification with triethylamine. BOC-protecting group was removed with trifluoracetic acid (TFA) solution and Z-protecting group—with catalytic hydrogenation. The product was extracted and purified by the method of preparative high-performance liquid chromatography (HPLC) on a reversed phase column.

Specification of the finished product:

| | | | | |
|---|---|---|---|---|
| Amino acid assay: | Glu | Asp | Ala | Pro |

| | | | |
|---|---|---|---|
| 1.10 | 1.01 | 1.00 | 1.10 |

peptide content: 98.56% (by HPLC, 200 nm);
thin layer chromatography (TLC)—individual; R_{f}=0.67 (acetonitrile-acetic acid-water 5:1:3);
moisture content: 7%;
pH of 0.001% solution: 4.24;
specific rotary power [α]_{D}²⁵:−78.9° (c=1.09, H₂O), “Polamat A”, Carl Zeiss Jena.

Synthesis is performed according to Example 2 and distinguished by the fact that at the C-end of the molecule benzyl ether of proline is used instead of benzyl ether of glycine.

Pharmaceutical composition is obtained according to Example 2.

### EXAMPLE 5

### Effect of Ala-Glu-Asp-Pro Tetrapeptide on the Growth of Cerebral Cortex Explants

The experiments are conducted on 73 fragments of cerebral cortex explants of 10-11-days' chicken embryos. The nutrient medium for cultivation consisted of 35% Eagle's solution, 25% calf foetal serum, 35% Hanks' solution, 5% chicken embryonic extract. The medium was also added glucose (0.6%), insulin (0.5 unit/ml), penicillin (100 unit/ml), glutamine (2 mM). Fragments of the cerebral cortex were placed in this medium and cultivated in Petri's dishes in a thermostat at 36.7° C. for 48 hours. The experimental medium is was added Ala-Glu-Asp-Pro tetrapeptide and Cortexin in the concentrations of 2, 10, 20, 50, 100, 200, 400 ng/ml. Square index (SI) was considered the criterion of biological activity. It was counted as the ratio between the total explant square including the growth zone and the initial square of the cerebral cortex fragment and. Significance of differences between the average values of SI was assessed by Student's t-criterion. SI values were expressed per cent, the control SI value being taken for 100%.

The growth zone of the control cerebral cortex explants included short neurites, migrating glia and fibroblast cells.

Immediate influence of the substances upon the cerebral cortex fragments was investigated in the following experimental series.

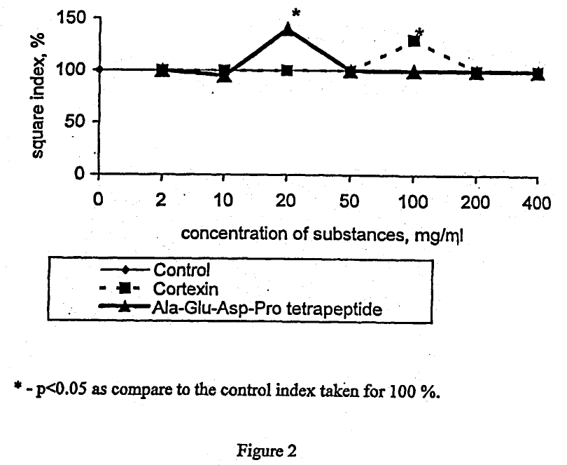

The nutrient medium of the explants of chicken embryonic cerebral cortex was added Cortexin in various concentrations. On the third day of cultivation, in the concentrations of 100 ng/ml a significant rise in the explant SI by 30±2% was noted in comparison with the control SI values. FIG. 2 exhibits the effect of Ala-Glu-Asp-Pro tetrapeptide on the growth of the cerebral cortex explants. No significant values of the cerebral cortex SI were registered in other Cortexin concentrations. Pronounced stimulation of the development of the cerebral cortex explants was revealed by applying Ala-Glu-Asp-Pro tetrapeptide in the concentration of 20 ng/ml when SI of the experimental explants was higher by 40±7% than that of the control cerebral cortex fragments.

Investigation of the cerebral cortex explants by longer periods of cultivation—up to 7 days—demonstrated analogous neurite-stimulating effects in the same concentrations. In certain cases, a statistically insignificant decrease in explant SI was noted, probably, due to the retraction of nerve fibres by longer cultivation periods.

Thus, regarding the cerebral tissues, a decrease in the threshold of effective concentrations of Ala-Glu-Asp-Pro tetrapeptide was registered in comparison with Cortexin. For instance, Cortexin stimulated the fragments of cultivated cerebral cortex in the concentration of 100 ng/ml, while the tetrapeptide—in the concentration of 20 ng/ml. This evidences a more expressed and directed action of Ala-Glu-Asp-Pro tetrapeptide upon the cerebral cortex neurones.

The performed studies and experiments suggest that the peptides obtained by the proposed method and pharmaceutical compositions on their basis possess a tissue-specific activity, i.e. they exert an effect upon the very tissues whose amino acid composition serves as the basis for their acquisition.

### EXAMPLE 6

### Synthesis of Lys-Glu-Asp-Ala Tetrapeptide

1. Product name: lysyl-glutamyl-aspartyl-alanine.
2. Structural formula: H-Lys-Glu-Asp-Ala-OH 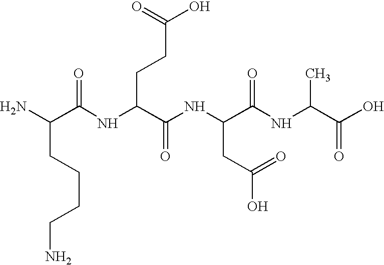
3. Molecular formula without ion pair: C₁₈H₃₁N₅O₉.
4. Molecular weight without ion pair 461.48.
5. Ion pair: acetate.
6. Appearance: white amorphous powder without smell.

7. Method of synthesis: the peptide is obtained by a classical method of synthesis in a solution by the following scheme:

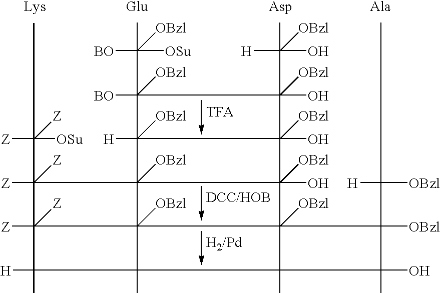

| | |
|---|---|
| Z | -benzyloxycarbonyl group; |
| BOC | -tert.butyloxycarbonyl group; |
| OSu | -N-oxysuccinimide ester; |
| OBzl | -benzyl ester; |
| DCC | -N,N′-dicyclohexylcarbodiimide; |
| HOBT | -N-oxybenzotriazol. |

N,N′-dimethylformamide was used as a solvent. When adding aspartic acid, the defence of α-COOH group was applied by salification with triethylamine. BOC-protecting group was removed with trifluoracetic acid (TFA) solution and Z-protecting groups—with catalytic hydrogenation. The product was extracted and purified by the method of preparative high-performance liquid chromatography (HPLC) on a reversed phase column.

Properties of the finished product:

| | | | | |
|---|---|---|---|---|
| amino acid analysis | Lys | Glu | Asp | Ala |

| | | | |
|---|---|---|---|
| 0.97 | 1.02 | 1.01 | 1.00 |

peptide content 98.75% (by HPLC, 220 nm);
thin layer chromatography (TLC)—individual, R_{f}=0.71 (acetonitrile-water 1:1);
moisture content: 7%;
pH of 0.001%-solution: 5.54;
specific rotary power [α]_{D}²³:
−28.0° (c=1.0; H₂O), “Polamat A”, Carl Zeiβ Jena.

Synthesis is performed according to Example 2 and distinguished by the fact that at the C-end of the molecule benzyl ether of alanine is used and at the N-end of the molecule oxysuccinimide ester of N^{α},N^{α}-dibenzyloxycarbonyl lysine is used instead of oxysuccinimide ester of N-benzyloxycarbonyl alamine.

Pharmaceutical composition is obtained according to Example 2.

### EXAMPLE 7

### Effect of Lys-Glu-Asp-Ala Tetrapeptide on the Intensity of Protein Synthesis in a Monolayer Hepatocyte Culture of Rats of Different Age

Intensity of the protein synthesis was investigated in the hepatocyte culture of rats aged 4, 8 and 18 months.

To isolate hepatocytes, rat liver was perfused with calcium-free Hanks' solution added 0.5 mM of EDTA and then 0.05% collagenase solution in Medium 199. The cellular suspension was filtered out and centrifuged. The hepatocyte suspension in the concentration of 5×10⁵ was introduced into Petri's dishes, their bottoms surfaced with collagen-covered glass. Applied Medium 199 contained no bovine serum but was added 0.2 mg/ml of albumen and 5 μg/ml of insulin. The dishes with glass-covered bottoms were placed in a thermostat at 37° C., aerated and added CO₂. In 2 hours, the glasses with adhered cells were washed and the medium was changed for a similar one. Twenty-four hours later and after washing the cultures, protein synthesis in them was investigated. Within 24 hours monolayer cultures with densely seated hepatocytes were formed in the cellular suspension at the above given concentration.

Protein synthesis was assessed by [³H]-leucin inclusion regarding the standard errors for a free marked leucin pull in the same culture. The molar activity of the applied leucin equalled 150 Ci/mM. Incubation with marked leucin took 10 minutes. After the incubation the cultures containing marked leucin were washed with the medium and treated with cold (4° C.) sulphuric acid for 90 minutes to isolate non-included leucin. The same culture was rinsed with ethyl alcohol, after which proteins were dissolved with hyamine. Radioactivity of the pull of free intracellular leucin and cellular proteins (in hyamine fraction) after adding the corresponding scintillators was measured on a radioactivity counter SL-30.

The intensity of protein synthesis was calculated by the formula
I_{corr}=Iᵢ×Pₐᵥ/Pᵢ(cpm), where

I_{corr}—inclusion of leucin regarding the standard errors of free leucin pull, Iᵢ—measured radioactivity of the proteins for i-culture, Pₐᵥ—mean radioactivity of proteins and pull for the cultures studied in this experiment, Pᵢ—total radioactivity of proteins and pull of the same culture.

Hepatocyte cultures were incubated with Lys-Glu-Asp-Ala tetrapeptide in the concentration of 0.005 μg/ml during 4 hours.

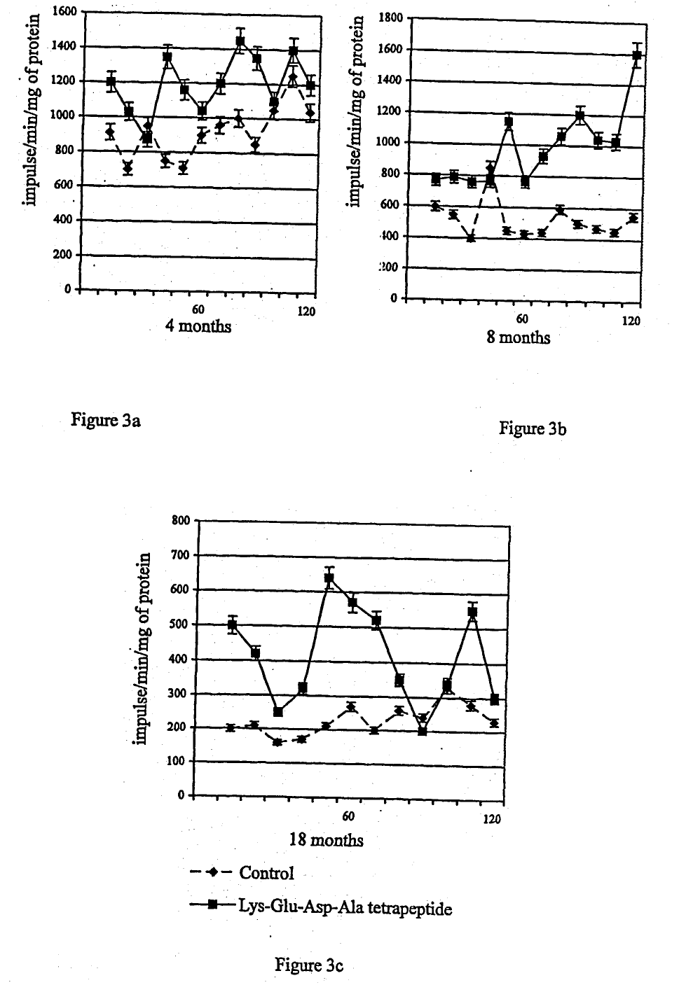

FIG. 3(a, b, c) demonstrates the effect of Lys-Glu-Asp-Ala tetrapeptide on the protein synthesis kinetics in hepatocyte monolayer culture of rats of different age.

The level of protein synthesis in the hepatocyte cultures was found to decrease with age (FIG. 3a, b, c). Addition of Lys-Glu-Asp-Ala tetrapeptide to the culture raised the level of protein synthesis in hepatocytes of rats of different age. Thereby, the strongest effect was observed in the cells of older animals. Besides, the amplitude of synthesis oscillations increased considerably in the hepatocytes of older rats, which enabled a conclusion on a raised degree of the cell population activity synchronisation (FIG. 3a, b, c).

### EXAMPLE 8

### Effect of Lys-Glu-Asp-Ala Tetrapeptide on the Development of Liver

The experiments were carried out in 53 liver fragments of 10-11 days old chicken embryos. Nutrient medium for the explant cultivation consisted of 35% of Eagle's solution, 25% of foetal calf serum, 35% of Hank's solution and 5% of chicken embryonic extract. The mixture is added glucose (0.6%), insulin (0.5 unit/ml), penicillin (100 unit/m) and glutamine (2 mM). The liver fragments were placed in this medium and cultivated in Petri's dishes in a thermostat at 36.7° C. during 48 hours. Lys-Glu-Asp-Ala tetrapeptide was added to the experimental medium in the concentrations of 2, 10, 20, 50, 100, 200 and 400 ng/mL Square index (SI) was taken for a biological activity criterion and calculated as a correlation of the total explant square including the growth zone to the initial square of a liver fragment. The SI values were expressed percent, the control SI value taken for 100%.

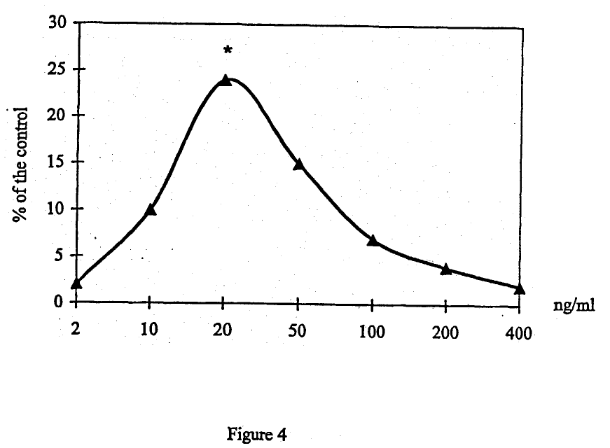

FIG. 4 demonstrates the effect of Lys-Glu-Asp-Ala tetrapeptide on the development of liver explants.

In 24 hours of cultivation, the explants on a collagen lining were found to lie flat Proliferating and migrating cells started to move along the explant periphery. By the tetrapeptide concentration of 20 ng/ml on the third day of the cultivation, a significant increase in the explant SI by 24% was observed as compared to the control value (FIG. 4). In case of longer terms of the liver explant cultivation (up to 7 days), an analogous stimulating effect of the tetrapeptide in the same concentration was revealed.

Consequently, Lys-Glu-Asp-Ala tetrapeptide exerted a tissue-specific effect upon the liver tissue expressed in the explant growth stimulation.

The conducted studies and experimental series enable the following conclusion: the peptides obtained by the claimed method and pharmaceutical compositions containing as their active bases these peptides or their salts possess a tissue-specific activity, i.e. they exert an action on the very tissues, whose amino acid composition serves the basis for their acquisition. Moreover, it is possible to create pharmaceuticals normalising the functions of various organs and tissues and containing the peptides with tissue-specific activity obtained by the claimed method.

### REFERENCES

1. Patent of the Russian Federation No. 1112606 “Method of obtaining an immunomodulator from the thymus”.—1982.
2. Patent of the Russian Federation No. 944191 “Method of obtaining a substance possessing an antitumour effect”.—1980.
3. Patent of the Russian Federation No. 1417244 “Method of obtaining a substance restoring the prostate function”.—1986.
4. Patent of the Russian Federation No. 2104702 “Method of obtaining from animal raw material of a complex of biologically active polypeptides normalising the brain functions, a pharmacological composition and its application”.—1996.
5. Patent of the Russian Federation No. 2073518 “Substance restoring the retinal function”.—1993.
6. Morazov V. G., Khavinson V. Kh. Peptide bioregulators (25-years' experience of experimental and clinic studies).—Nauka, St. Petersburg.—1996.—74 p.
7. Yakubke Kh.-D., Eshkeit Kh. Amino acids, peptides, proteins: translated from German.—Mir, Moscow.—1985.—456 p.
8. Khavinson V. Kh., Zhukov V. V., Deigin V. I., Korotkov A. M. Effect of Thymalin and synthetic peptide of the thymus on the activity of the enzymes of purine nucleotide metabolism in thymocytes.//Abstr. Conference “Biochemistry—to medicine”.—Leningrad.—1988.—PP. 198-199.
9. Schlesinger D. H., Goldstein G. The amino acid sequence of thymopoietin II//Cell.—1975.—Vol. 5, No. 4.—PP. 361-365.
10. Audbya T., Scheid M. P., Goldstein G. Contrasting biological activities of thymopoietin and splenin, two closely related polypeptide products of thymus and spleen//Proc. Natl. Acad. Sci USA.—1984.—Vol. 81, No. 9.—PP. 2847-2849.
11. Fridkin M., Najjar V. A Tuftsin: its chemistry, biology, and clinical potential//Crit. Rev. Biochem. Mol. Biol.—1989.—Vol. 24, No. 1.—PP. 140.
12. Pokrovsky V. I., Suleimanov A. K., Lebedev V. V. et al. Immunorehabilitating effect of thymogexin in the treatment of patients with chronic brucellosis.//Therapeutic archive.—1992.—Vol. 64, No. 11.—PP. 22-26.
13. Pigareva N. V., Kalinin N. M, Simbirtsev A. S. Characteristic of the immunologic action of synthetic peptide <<bestim>>//Medical immunology.—1999.—Vol. 1, No. 34.—127 p.
14. Teitelbaum D., Aharoni R., Arnon R., Sela M. Specific inhibition of the T-cell response to myelin basic protein by the synthetic copolymer Cop 1//Proc. Natl. Acad. Sci. USA.—1988.—Vol. 85, No 24.—PP. 9724-9728.

1. A method of obtaining a peptide with tissue-specific activity comprising amino acid analysis of an acetic extract from an animal tissue, selection of amino acids prevailing in the studied tissue, synthesis of a central link of the peptide and attachment to its both ends of amino acids prevailing among the remaining amino acids in the studied tissue.

2. The method of claim 1, wherein the amino acid analysis is conducted on an acetic extract from epiphysis tissue.

3. The method of claim 1, wherein the amino acid analysis is conducted on an acetic extract from cerebral cortex tissue.

4. The method of claim 1, wherein the amino acid analysis is conducted on an acetic extract from liver tissue.

5. The method of claim 1, wherein the central link is presented by glutamic (Glu) and aspartic acids (Asp).

6. The method of claim 1, wherein the amino acids are joined to the central link a N- and C-ends.

7. A pharmaceutical composition possessing a tissue-specific activity and containing an active base and a pharmaceutically admissible carrier, wherein the composition contains as its active base an effective quantity of a peptide obtained by the method of claim 1.

8. The composition of claim 7, which contains a salt of the amino group, of carboxyl group, or a salt of an organic or inorganic cation.

9. A pharmaceutical composition possessing a tissue-specific activity and containing an active base and a pharmaceutically admissible carrier, wherein the composition contains as its active base an effective quantity of a peptide obtained by the method of claim 2.

10. A pharmaceutical composition possessing a tissue-specific activity and containing an active base and a pharmaceutically admissible carrier, wherein the composition contains as its active base an effective quantity of a peptide obtained by the method of claim 3.

11. A pharmaceutical composition possessing a tissue-specific activity and containing an active base and a pharmaceutically admissible carrier, wherein the composition contains as its active base an effective quantity of a peptide obtained by the method of claim 4.

12. A pharmaceutical composition possessing a tissue-specific activity and containing an active base and a pharmaceutically admissible carrier, wherein the composition contains as its active base an effective quantity of a peptide obtained by the method of claim 5.

13. A pharmaceutical composition possessing a tissue-specific activity and containing an active base and a pharmaceutically admissible carrier, wherein the composition contains as its active base an effective quantity of a peptide obtained by the method of claim 6.

14. The composition of claim 9, which contains a salt of the amino group, of carboxyl group, or a salt of an organic or inorganic cation.

15. The composition of claim 10, which contains a salt of the amino group, of carboxyl group, or a salt of an organic or inorganic cation.

16. The composition of claim 11, which contains a salt of the amino group, of carboxyl group, or a salt of an organic or inorganic cation.

17. The composition of claim 12, which contains a salt of the amino group, of carboxyl group, or a salt of an organic or inorganic cation.

18. The composition of claim 13, which contains a salt of the amino group, of carboxyl group, or a salt of an organic or inorganic cation.
